(19) United States
(12) Patent Application Publication
(10) Pub. No.: US 2005/0147965 A1
(43) Pub. Date: Jul. 7, 2005

(54) Compositions and methods for detecting reverse transcriptase in a sample

(76) Inventors: Zhandong Don Zhong, Thousand Oaks, CA (US); Martha Garrity, San Clemente, CA (US)

Correspondence Address: FOLEY & LARDNER, P.O. BOX 80278, SAN DIEGO, CA, 92138-0278, US

(21) Appl. No.: 10/750,092

(22) Filed: Dec. 31, 2003

(51) Int. Cl. C12Q 1/70; C12Q 1/68

(52) U.S. Cl. 435/5; 435/6

(57) ABSTRACT

The present invention provides methods for detecting the presence and enzymatic activity of reverse transcriptase (RT) in a sample. The methods generally involve conducting a reverse transcriptase PCR assay in the presence of labeled deoxynucleotides. The deoxynucleotides are incorporated into a molecular structure or complex containing the RNA template and the extending cDNA primer. In one embodiment the deoxynucleotides are labeled with a detectable moiety. A capture moiety can also be included to immobilize the complex on a surface after completion of the reaction and facilitates detection of the molecular structure, and therefore the presence of reverse transcriptase. In one embodiment the detectable moiety is a chemiluminescent moiety, such as an acridinium dye, and the assay is determined by stimulating chemiluminescence from the detectable moiety and detecting light emitted. In various embodiments the assays are also useful for determining the sub-type of reverse transcriptase present in a sample, or for screening for anti-retroviral lead compounds. Also disclosed are kits for conducting the assay.

### FIELD OF THE INVENTION

The present invention relates to assays for detecting the presence of reverse transcriptase in a sample.

### BACKGROUND OF THE INVENTION

The following description of the background of the invention is provided simply as an aid in understanding the invention and is not admitted to describe or constitute prior art to the invention.

Retroviruses are viruses whose genomes consist of RNA. These viruses include important members such as HIV-1 and HIV-2, which are the viral agents that cause AIDS. Retroviruses are also responsible for a variety of other diseases, including leukemias and lymphomas in humans and animals. A typical “minimal” retrovirus consists of an outer envelope which was derived from the plasma membrane of its host; copies of an envelope protein, which are embedded in the lipid bilayer of its envelope, a capsid, a protein shell containing two molecules of RNA, and molecules of the enzyme reverse transcriptase.

Considerable efforts have been made in the development of techniques for quantifying retrovirus load with particular attention to the detection of type 1 human immunodeficiency virus (HIV-1). HIV-1 is a retrovirus that causes acquired immunodeficiency syndrome (AIDS).

There exists a need in the art for methods of detecting and quantitating the presence of retroviruses that can detect variant or other highly divergent viruses, and that are able to distinguish RNA of a competent genome from that of a defective one. In addition, it would be desirable to have methods where the results could not be nullified by PCR contamination or RNA degradation due to RNA extraction and sample manipulation.

### SUMMARY OF THE INVENTION

The present invention provides methods for detecting the presence and enzymatic activity of reverse transcriptase (RT) in a sample. The methods generally involve conducting a reverse transcriptase PCR assay in the presence of one or more labeled deoxynucleotides. The one or more deoxynucleotides are incorporated into a molecular structure or complex containing the RNA template and the extending cDNA primer. The one or more deoxynucleotides are labeled with a detectable moiety and, in one embodiment, also a capture moiety. There may also be present “free” deoxynucleotides, that are not labeled with either the detectable or capture moieties. In one embodiment the detectable moiety is a chemiluminescent moiety, such as an acridinium dye, and the assay is determined by stimulating chemiluminescence from the detectable moiety incorporated into the extending DNA primer and detecting light emitted. In various embodiments the assays are also useful for determining the sub-type of reverse transcriptase present in a sample, or for screening for anti-retroviral lead compounds. When used, the capture moiety immobilizes the molecular structure or complex on a surface or facilitates removal of the molecular structure from the reaction mixture after completion of the reaction, thereby enabling its detection as an indicator of the presence or activity of reverse transcriptase. Also disclosed are kits for conducting the assay.

In a first aspect the present invention provides methods of detecting the presence of reverse transcriptase in a sample. The methods involve contacting the sample with a reaction mixture containing an RNA template, a DNA primer, one or more deoxynucleotide triphosphates (dNTP) labeled with a detectable moiety and, optionally, one or more deoxynucleotide triphosphates not labeled with a detectable moiety. The reaction mixture is incubated under conditions suitable to generate a molecular structure that contains an extended DNA primer containing the detectable moiety when reverse transcriptase is present in the sample. The detectable signal is then detected as an indication of the presence of reverse transcriptase in the sample. In one embodiment at least one of the RNA template, DNA primer, deoxynucleotide triphosphate labeled with a detectable moiety, and deoxynucleotide triphosphate not labeled with a detectable moiety contains a capture moiety. Thus, the capture moiety can be incorporated into the extending DNA primer and used to separate the molecular complex from the reaction mixture. In one embodiment the detecting step involves detecting the presence of the molecular structure containing an incorporated detectable moiety. The molecular structure can contain the RNA template, the DNA primer, and one or more deoxynucleotide triphosphates labeled with the detectable moiety and, optionally, one or more deoxynucleotide triphosphates not labeled with the detectable moiety.

By “reverse transcriptase” is meant an RNA-dependent DNA polymerase, which converts genetic material from RNA to DNA. In one embodiment the reverse transcriptase is from a retrovirus. By “RNA template” is meant an RNA molecule whose structure is a pattern for the synthesis of a complementary cDNA molecule by reverse transcriptase. Thus, in general where the RNA template has A, U, C, and G, the complementary cDNA molecule will generally have, respectively, T, A, G, and C. By a “DNA primer” is meant an oligonucleotide that can be extended by a DNA polymerase, or a functional fragment of a DNA polymerase. The primer serves as the starting point for the creation of the extending DNA primer.

In some embodiments the RNA template and DNA primer are parts of the same molecule. For example the RNA template can be poly(rA), and the DNA template can be poly-T which is present at the 3′ end of the RNA template. The poly-T sequence folds back on the poly(rA) and hybridizes with a portion of it, thus providing a DNA primer on the RNA template.

A “capture moiety” refers to a portion of a molecule that can be used to separate the molecule from a solution. Thus, a moiety that has a binding affinity for another molecule can be a capture moiety. The binding affinity need only be sufficient to collect the capture moiety (and consequently the molecular structure or complex attached to it) from a solution. Suitable capture moieties include, but are not limited to, biotin, streptavidin, streptavidin agarose, digoxigenin, and various fluorescent compounds such as, for example, fluorescein and 5(6)-carboxy-fluorescein-N-hydroxysuccinimide ester (FLUOS), rhodamine, aminomethylcoumarin acetic acid, cyanine dyes (e.g., Cy3), and commercially available products such as CaptAvidin™ agarose (Molecular Probes, Eugene, Oreg.), Captivate™ ferrofluid magnetic particles (Molecular Probes, Eugene, Oreg.). In one embodiment the capture moiety is a molecule that can be bound by an antibody. The capture moiety can also be a particle or portion of a molecule that is pulled from solution by a force such as a magnetic attraction. For example, the capture moiety can be a magnetic micro-bead or a molecule attached to a micro-bead. The molecular structure or complex can be separable from the reaction mixture through the capture moiety. Thus, in one embodiment the capture moiety is biotin, which can be removed from solution by contacting the reaction mixture with magnetic particles coated with streptavidin. After mixing, the magnetic particles are separated from the solution and the quantity of signal present on the magnetic particles determined.

By “molecular structure” is meant one or more nucleic acid and other molecules bound together, non-covalently. In one embodiment the molecular structure is a complex containing the RNA template hybridized to the extending cDNA primer. The molecular structure also contains the detectable moiety, which can be carried by one or more nucleotides contained in the structure. The molecular structure also contains a capture moiety (when used) and can also contain the reverse transcriptase, when present. The capture moiety can be carried by one or more of the nucleotides, the RNA template, or the cDNA primer. By an “extending DNA primer,” “extended primer” or “extending cDNA primer” is meant a strand of DNA to which has been added at least one deoxyribonucleotide based on the structure of the RNA template. By “reaction mixture” is meant the mixture of the RNA template, DNA primer, nucleotides, and other components of the reverse transcriptase reaction. In various embodiments the reaction mixture also contains buffers, metal ions (e.g., divalent metal ions), enzyme inducers, and other components that facilitate the enzymatic reaction or detection of its products.

In one embodiment the detectable moieties are acridinium moieties and the detectable signal is the emission of light produced by a chemiluminescent reaction. Acridinium moieties include, but are not limited to, acridinium esters such as C₂NHS and acridinium sulfonamides, both of which can be readily attached to nucleic acids and nucleotides.

In another embodiment the method further involves separating the molecular structure or complex from the sample before generating the detectable signal. The separation can be performed by utilizing the capture moiety contained in the molecular structure. For example, when the capture moiety is biotin that has been attached to a deoxynucleotide triphosphate that is incorporated within the molecular structure or complex, the molecular structure or complex can be separated from the reaction mixture by contacting the reaction mixture with avidin immobilized on a surface of a solid phase. Alternatively, streptavidin, streptavidin agarose, CaptAvidin™ agarose (Molecular Probes, Eugene, Oreg.), Captivate™ ferrofluid magnetic particles (Molecular Probes, Eugene, Oreg.), can also be used to remove the biotinylated molecular structure or complex from the reaction mixture. Any other capture moiety can be used, as long as it can be removed from the reaction mixture or at least isolated within the reaction mixture. Of course, any of the above molecules can be used as the capture moiety and removed with a corresponding molecule having affinity for the capture moiety. In other embodiments the molecular structure can be removed from the reaction mixture by chromatographic methods, such as gel filtration (or “size exclusion”) chromatography.

In yet another embodiment, deoxynucleotide triphosphates that have not been incorporated into the extending DNA primer (unreacted dexoynucleotide triphosphates) are removed from the reaction mixture prior to generating the detectable signal. Thus, instead of removing the detectable moiety that generates the detectable signal, deoxynucleotides that have not been incorporated into the molecular structure are removed so as not to interfere with the detection of signal from those detectable moieties contained in the molecular structure or complex. In one embodiment the detectable signal is from a chemiluminescent reaction which causes the emission of light from the detectable moiety. When the detectable moiety is acridinium, the signal is generated by the addition of a dilute acid and hydrogen peroxide to the reaction mixture.

In various embodiments the RNA template is made of homopolymeric and/or heteropolymeric RNA, and the deoxynucleotide triphosphates are dCTP, dGTP, dATP, and dTTP. In one embodiment the reaction mixture further comprises one or more divalent metal ions present at a concentration of about 5 mM, and the divalent metal ions can be selected from the group consisting of magnesium and manganese. In various other embodiments the divalent metal ions can be present, for example, at 2-50 mM, or 3-20 mM, 10 mM, or 15 mM, or 20 mM, or 25 mM. When used, the capture moieties can be haptens. In one embodiment the DNA primer contains the capture moiety and in another embodiment the RNA template contains the capture moiety. In one embodiment the detectable moieties are incorporated into the cDNA by extension of the cDNA primer under conditions suitable to preserve the signal of the detectable moiety. In various embodiments the detectable moiety is an acridinium moiety, and the signal is the emission of light. In one embodiment the deoxynucleoside triphosphates labeled with acridinium moieties have the formula:
TP-Sugar-Px-L-Acr
wherein:
   TP is a triphosphate group attached to the 5′ position of the sugar;
   sugar is a sugar moiety;
   Px is a purine, pyrimidine, or 7-deazapurine, wherein Px is attached to the 1′ position of the sugar moiety through the N1 position when Px is a pyrimidine or through the N9 position when Px is a purine or a 7-deazapurine;
   L is a linear or branched hydrocarbylene or heterocarbylene linker of at least one carbon atom, wherein L is covalently attached to Acr at one end of L, and at another end to Px through position C5 or C6 of Px when Px is a pyrimidine, or through position C8 of Px when Px is a purine, or through position C7 or C8 of Px when Px is a 7-deazapurine;
   Acr is an acridinium moiety; and the detectable signal is the emission of light. As used herein, “about” means plus or minus 10%.

In one embodiment the linker, L, is a linear hydrocarbylene or heterocarbylene linker of at least one carbon atom, and in another embodiment the linker, L, is a linear alkenylene or heteroalkenylene linker containing at least 3 carbon atoms. Thus, in various embodiments the linker L is —CH₂—CH═CH—CH₂—, —CH═CH—CH₂—NH—, —NH(CH₂)₆NH—, —NH(CH₂CH₂O)ₙNH—, C≡C—CH₂NH—, or —CH₂—C≡C—CH₂—. The acridinium moiety is stable under the conditions of reverse transcription, and the acridinium moiety is detectable after incorporation into the cDNA molecule by the emission of light.

In another aspect the present invention provides a molecular structure of an RNA template hybridized to a DNA molecule and which has a deoxynucleotide linked to a detectable moiety. The link can be a covalent bond. In one embodiment the RNA template also has a capture moiety, but the DNA molecule can also contain the capture moiety. In various embodiments the detectable moiety is an acridinium moiety.

In another aspect the present invention provides a kit containing an RNA template, a DNA primer complementary to a region of the RNA template and of length sufficient to form a stable template-primer hybrid molecule with the RNA template, and a deoxynucleotide triphosphate labeled with a detectable moiety. In one embodiment the RNA template and DNA primer are parts of the same molecule, which can be a poly-(rA) with a poly-T sequence attached to one end. The poly-(dT) sequence can hybridize to the poly-(rA) sequence by the nucleic acid looping around on itself, and thus the RNA template and DNA primer can be parts of the same molecule. In various embodiments the length of the poly-(dT) can be T₁₀₋₅₀ or T₁₂₋₃₅ or T₁₂₋₃₀ or T₁₂₋₁₈ or T₂₅. The kit can also contain buffers for conducting a reverse transcriptase assay, and the buffers can contain a divalent metal ion at a concentration of about 5 mM. The detectable moiety can be an acridinium moiety. In one embodiment the deoxynucleotide triphosphate can further contain a capture moiety.

In another aspect the present invention provides methods for determining a sub-type of reverse transcriptase present or absent in a sample. The methods involve contacting a sample to be tested with a binding molecule (e.g., an antibody and aptamer) specific for a sub-type of reverse transcriptase, and contacting the sample with a reaction mixture as described above. Whether a molecular structure containing an extended DNA primer that has the detectable moiety is present is determined, and it is thereby determined whether the sub-type of reverse transcriptase is present or absent in the sample. If the sub-type of reverse transcriptase that the binding molecule is specific for is present in the sample, little or no signal will be generated by that sub-type of reverse transcriptase. The presence or absence of the sub-type of reverse transcriptase in the sample is therefore determined. In one embodiment the binding molecule is immobilized on a surface. The binding of the binding molecule inhibits the activity of the particular sub-type of reverse transcriptase causing the detectable signal to be diminished or absent due to failure of detectable signal to be incorporated into the extending DNA primer.

By “binding molecule” is meant a molecule or fragment thereof that has a specific and substantial affinity for a target molecule, and that does not bind generally with molecules other than the target molecule. The target molecule can be a sub-type of reverse transcriptase. The binding of a binding molecule must be specific enough such that the act of binding can serve as a reliable identifier of the target molecule. In one embodiment the percent confidence of the identity of the target based on binding of the binding molecule is at least 90%. In other embodiments the percent confidence is at least 95% or 97% or 98% or 99%. The act of binding to the sub-type of reverse transcriptase may also serve to block the activity of the sub-type. Thus, in those embodiments, the absence of signal produced is an identifier. In some embodiments the binding molecule is an antibody or fragment thereof. For example, the binding molecule can be only the F_{c} portion of an antibody, or only the Fₛᵥ binding region of an antibody. The binding molecule and molecular structure it is bound to can be separated from the reaction mixture by attaching a capture moiety to one member of the molecular structure, including the binding molecule itself.

In another aspect the present invention provides a method of screening for anti-retroviral lead compounds. The methods involve contacting a compound to be tested for anti-retroviral activity with a sample of reverse transcriptase, contacting the compound and reverse transcriptase with a reaction mixture as described herein, determining whether a molecular structure is generated that contains an extended DNA primer having the detectable moiety; and thereby screening the compound for anti-retroviral activity. The effectiveness of the compound can be inferred based on the measurement of reverse transcriptase activity and reduction in reverse transcriptase activity caused by the presence of the compound. The detectable signal is measured and the screening conducted for anti-retroviral lead compounds. The molecular structure can also contain a capture moiety, as described above. By “lead compound” is meant a compound that exhibits pharmacological or biochemical properties which suggest its value as a starting point for drug development.

In another aspect the present invention provides methods for monitoring anti-viral therapy. The methods involve contacting a sample from a patient undergoing anti-viral therapy with a reaction mixture as described above, generating a molecular structure as described above when reverse transcriptase is present in the sample, and measuring the detectable signal and thereby monitoring the anti-viral therapy. By “monitoring anti-viral therapy” is meant that the progress of therapy is reviewed with respect to success or failure of the therapy.

In another aspect the present invention provides methods of detecting the presence of reverse transcriptase in a sample. The methods involve contacting the sample with a reaction mixture containing an RNA template, a DNA primer, and one or more deoxynucleotide triphosphates labeled with a detectable moiety. One of the RNA template or DNA primer is immobilized on a solid phase. The reaction mixture is incubated under conditions suitable to generate a molecular structure having an extended primer that carries the detectable moiety when reverse transcriptase is present in the sample. Reverse transcriptase is then detected in the sample.

In another aspect the present invention provides novel acridinium-containing compositions. The compositions contain a molecular structure or complex containing any of the following: an RNA template and an acridinium-containing cDNA complementary thereto; a complex of an RNA template and a complementary cDNA containing an acridinium moiety and a capture moiety; an RNA template labeled with a capture moiety and a cDNA complementary to the RNA template that contains an acridinium ester; an RNA template, a DNA primer containing a capture moiety, and deoxynucleotide triphosphates labeled with an acridinium moiety; a RNA template containing a capture moiety, a DNA primer, and deoxynucleotide triphosphates labeled with an acridinium moiety.

The summary of the invention described above is non-limiting and other features and advantages of the invention will be apparent from the following detailed description of the invention, and from the claims.

### BRIEF DESCRIPTION OF THE FIGURES

FIG. 1 provides a graphical illustration of the synthesis of an acridinium-labeled deoxyuridine triphosphate.

FIG. 2 provides a graphical illustration of a reverse transcriptase (RT) assay curve for rHIV. The graph shows chemiluminescent activity from an extended cDNA primer synthesized according to the present invention.

FIG. 3 provides some embodiments of acridinium esters and acridinium sulfonamides useful in the present invention. FIG. 3a is the acridinium C₂NHS ester, 4-(2-sucinimidyl-oxycarbonylethyl)-phenyl-10-acridinium-9-carboxylate trifluoromethyl sulfonate. FIG. 3b is 1-methyl-acridinium ester, and FIG. 3c is 1-methyl-di-meta-fluoro-acridinium ester.

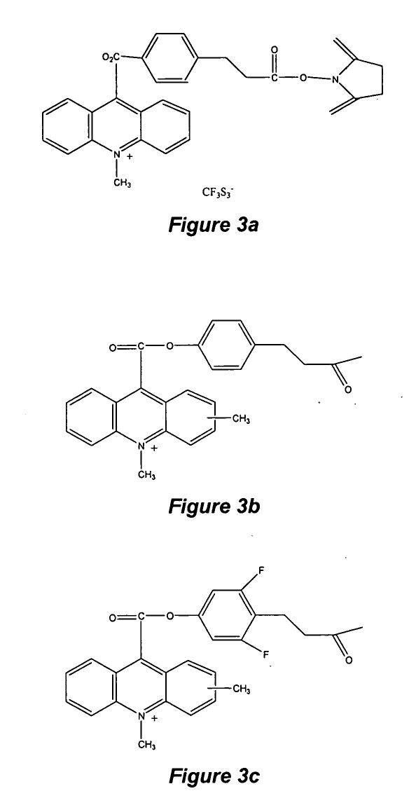

### DETAILED DESCRIPTION OF THE INVENTION

Chemiluminescence is the emission of light from a chemical reaction. In some embodiments chemiluminescence occurs at ambient temperatures, such as from about 20 C to about 30 C. There are enormous numbers of chemical reactions that produce light (i.e., are chemiluminescent), but a much smaller number that have sufficiently high efficiencies of chemiluminescence to be useful in molecular analysis.

Acridinium Esters

Aridinium esters have good chemiluminescence efficiencies and provide a detectable light production. The compounds can also be attached to nucleic acids without significant detrimental effect on their chemiluminescence properties.

Acridinium esters are of the “flash” type chemiluminescent reactions, where the addition of reagent causes the immediate emission of light over a period of milliseconds or seconds. A photon counting luminometer that uses a syringe or bellows pump type reagent injector is useful in the detection of flash-type luminescence. These types of injectors offer high reproducibility for injection function. Many commercial luminometers are available, including programmable models offering a simultaneous or sequential dispensing. Luminescence is conveniently measured in 96-well plates.

Attachment to Nucleic Acid

Attachment of acridinium esters to nucleic acids containing a primary amino group is carried out by dissolving the acridinum ester in a dry aprotic solvent such as dimethyl formamide and adding the solution to the nucleic acid in a suitable buffer. Suitable buffers include those that do not have amine groups, such as borate or bicarbonate buffers at a pH of between 7 and 10, although a pH of 8.5 is generally desirable for labeling. Excess acridinium ester label is easily removed by dialysis or gel filtration through a resin such as Sephadex G-10. Thus, in one embodiment the acridinium ester is 4-(2-sucinimidyl-oxycarbonylethyl)-phenyl-10-acridinum-9-carboxylate trifluoromethyl sulfonate, and has a molecular weight of 632.55.

In one embodiment of the invention the methods of the invention detect the enzymatic activity of reverse transcriptase (RT) as an indicator of the presence of a retrovirus in a biological sample. In the presence of RT under the conditions described herein, acridinium-labeled deoxynucleotides are incorporated into a growing cDNA chain during the extension of a DNA primer hybridized to an RNA template.

Acridinium-Labeled Deoxynucleoside Triphosphates

In various embodiments, acridinium-labeled deoxynucleoside triphosphates useful in the invention have the formula:
TP-Su-Px-L-ACR
wherein:
   TP is a triphosphate group attached to the 5′ position of Su;
   Su is a sugar moiety;
   Px is a pyrimidine, purine, or 7-deazapurine, wherein Px is attached to the 1′ position of Su through the N1 position when Px is a pyrimidine or through the N9 position when Px is a purine or a 7-deazapurine;
   L is a linear or branched hydrocarbylene or heterocarbylene linker of at least one carbon atom, wherein L is covalently attached to ACR at one end of L, and at another end to Px through position C5 or C6 of Px when Px is a pyrimidine, or through position C8 of Px when Px is a purine, or through position C7 or C8 of Px when Px is a 7-deazapurine; and
   ACR is an acridinium moiety.

Exemplary sugar moieties include glucose, fructose, ribose, ribulose, xylose, xylulose, galactose, streptose, hydroxystreptose, kanosamine, 3-amino-3-deoxy-D-ribose, D-glucosamine, and the like.

In one embodiment, the linker, L, is a linear hydrocarbylene or heterocarbylene linker of at least one carbon atom. “Hydrocarbylene” refers to divalent straight or branched chain hydrocarbyl groups including alkylene groups, alkenylene groups, alkynylene groups, cycloalkylene groups, heterocycloalkylene groups, cycloalkenylene groups, arylene groups, alkylarylene groups, arylalkylene groups, arylalkenylene groups, arylalkynylene groups, alkenylarylene groups, alkynylarylene groups, and the like. “Substituted hydrocarbylene” refers to hydrocarbylene groups further bearing one or more substituents such as hydroxy, alkyl, alkoxy (of a lower alkyl group), mercapto (of a lower alkyl group), cycloalkyl, substituted cycloalkyl, heterocyclic, substituted heterocyclic, aryl, substituted aryl, heteroaryl, substituted heteroaryl, aryloxy, substituted aryloxy, halogen, trifluoromethyl, cyano, nitro, nitrone, oxo, amino, amido, maleimido, succinimido, itaconimido, —C(O)H, acyl, oxyacyl, carboxyl, carbamate, sulfonyl, sulfonamide, sulfuryl, and the like.

An “alkylene” refers to divalent straight or branched chain hydrocarbyl groups having in the range of from 1-500 carbon atoms, and “substituted alkylene” refers to alkylene groups further bearing one or more substituents as set forth above. In other embodiments the alkylene is from 1-30 carbon atoms, or 1-20 carbon atoms, or 1-15 carbon atoms, or 1-10 carbo atoms. “Alkenylene” refers to divalent straight or branched chain hydrocarbyl groups having at least one carbon-carbon double bond, and typically having in the range of from 2-500 carbon atoms, and “substituted alkenylene” refers to alkenylene groups further bearing one or more substituents as set forth above. In other embodiments the alkenylene is from 1-30 carbon atoms, or 1-20 carbon atoms, or 1-15 carbon atoms, or 1-10 carbo atoms. “Alkynylene” refers to divalent straight or branched chain hydrocarbyl groups having at least one carbon-carbon triple bond, and typically having in the range of from 2-500 carbon atoms, and “substituted alkynylene” refers to alkynylene groups further bearing one or more substituents as set forth above. In other embodiments the alkynylene is from 1-30 carbon atoms, or 1-20 carbon atoms, or 1-15 carbon atoms, or 1-10 carbo atoms. “Cycloalkylene” refers to divalent ring-containing groups containing in the range of from 3 to about 20 carbon atoms, and “substituted cycloalkylene” refers to cycloalkylene groups further bearing one or more substituents as set forth above. “Heterocycloalkylene” refers to divalent cyclic (i.e., ring-containing) groups containing one or more heteroatoms (e.g., N, O, S, or the like) as part of the ring structure, and having in the range of from 1 to about 14 carbon atoms and “substituted heterocycloalkylene” refers to heterocycloalkylene groups further bearing one or more substituents as set forth above. “Cycloalkenylene” refers to divalent ring-containing groups containing in the range of from 3 to about 20 carbon atoms and having at least one carbon-carbon double bond, and “substituted cycloalkenylene” refers to cycloalkenylene groups further bearing one or more substituents as set forth above.

“Arylene” refers to divalent aromatic groups typically having in the range of 6 up to 14 carbon atoms and “substituted arylene” refers to arylene groups further bearing one or more substituents as set forth above. “Alkylarylene” refers to alkyl-substituted divalent aryl groups typically having in the range of about 7 up to 16 carbon atoms and “substituted alkylarylene” refers to alkylarylene groups further bearing one or more substituents as set forth above. “Arylalkylene” refers to aryl-substituted divalent alkyl groups typically having in the range of about 7 up to 16 carbon atoms and “substituted arylalkylene” refers to arylalkylene groups further bearing one or more substituents as set forth above. “Arylalkenylene” refers to aryl-substituted divalent alkenyl groups typically having in the range of about 8 up to 16 carbon atoms and “substituted arylalkenylene” refers to arylalkenylene groups further bearing one or more substituents as set forth above. “Arylalkynylene” refers to aryl-substituted divalent alkynyl groups typically having in the range of about 8 up to 16 carbon atoms and “substituted arylalkynylene” refers to arylalkynylene group further bearing one or more substituents as set forth above. “Alkenylarylene” refers to alkenyl-substituted divalent aryl groups typically having in the range of about 8 up to 16 carbon atoms and “substituted alkenylarylene” refers to alkenylarylene groups further bearing one or more substituents as set forth above. “Alkynylarylene” refers to alkynyl-substituted divalent aryl groups typically having in the range of about 8 up to 16 carbon atoms and “substituted alkynylarylene” refers to alkynylarylene groups further bearing one or more substituents as set forth above.

In one embodiment of the invention the linker, L, is a linear alkenylene or heteroalkenylene linker containing at least 3 carbon atoms. Examples of such presently preferred linkers include —CH₂—CH═CH—CH₂—, —CH═CH—CH₂—NH—, and the like.

In one embodiment the acridinium moieties are in a stabilized form such that they are compatible with the conditions of the reverse transcriptase assay, thereby allowing chemiluminescent measurement thereof upon incorporation into the cDNA copy.

The incorporation of one or more acridinium moieties into an extending DNA primer in accordance with the present invention allows the ultra-sensitive detection of and quantitation of reverse transcriptase. Thus, low levels of retroviral load can be detected and measured in the sample.

It was discovered unexpectedly that such ultra-sensitivity can be achieved according to the present invention in a single reverse transcription step without the need to amplify the cDNA product of the reverse transcription reaction. The cDNA synthesis conditions can readily be optimized from known protocols that include divalent metal cations and buffering agents in appropriate concentrations. Proper temperature is also desirable for effectively annealing the DNA primer with the RNA template and polymerization of the dNTP mixture while maintaining the stability of the acridinium-labeled deoxynucleoside triphosphates. Other conditions can readily be established to optimize a particular reverse transcriptase assay based on known protocols. The invention therefore functions without the need to use isotopic reagents in the reaction assays or reaction mixture, thereby eliminating the problem of disposal of such reagents. Furthermore, there is no secondary detection scheme necessary in the present invention as detection is performed directly through measurement of the extending primer, thereby eliminating the need for an enzyme-mediated spectrophotometric reaction or for hybridization of the extended cDNA primer to a DNA probe carrying a label, or for any need to amplify the cDNA product. In those embodiments of the present methods using acridinium labeled dNTPs, the incorporation of multiple acridinium labels into the extending cDNA copy during reverse transcription allows ultra-sensitive detection of the presence and activity of reverse transcriptase. Detection of the acridinium label does not require a secondary detection scheme, nor an excitation source such as the use of fluorescence. Detection is achieved by simply contacting the reaction mixture with, for example, dilute acid and alkaline hydrogen peroxide. The simplicity of the present methods therefore allows the ready automation of the assays.

In one embodiment the extending DNA primer containing one or more acridinium moieties is separated from excess acridinium triphosphate reagent prior to triggering for detection. Many methods are available for achieving the separation such as, for example, use of a capture moiety, solid phase strategies, size exclusion chromatography, etc. Solid-phase agents are capable of capturing and separating the cDNA product. Examples of these solid-phase agents include beads composed of polymeric materials, paramagnetic beads, micro-titer plates, glass or plastic slides, membranes, and the like.

In one embodiment the solid phase strategy involves a deoxynucleotide triphosphate (dNTP) that is labeled with a capture moiety and is included in the reaction mixture, which also contains acridinium-labeled dNTP. The capture moiety is a hapten that is incorporated into the extending cDNA primer with the dNTP that it labels, along with the acridinium moiety that also labels the dNTP. This allows the extending cDNA primer to be captured on a solid phase through the specific interaction of the hapten with a binding molecule immobilized on the solid phase specific for the hapten. Such interactions are exemplified by binding between an antigen and an antibody, a ligand and a receptor or an aptamer, and biotin/streptavidin or avidin. The capture cDNA can then be separated from unreacted excess acridinium labels prior to triggering and detection.

In another embodiment a capture moiety is attached to the DNA primer without the use of dNTPs labeled with a capture moiety in the reaction mixture. Rather, the DNA primer is tagged with the capture moiety, for example at the 5′ end or on one of the nucleotides of the primer. The extending cDNA primer containing the capture moiety and label incorporated through one or more labeled-dNTPs is captured on a solid phase after the reverse transcriptase reaction, thus permitting separation of the labeled cDNA strand from the reaction mixture.

In another embodiment the capture moiety is attached to the RNA template, for example at the 3′ end or 5′ end of the template or in one of the incorporated nucleotides. The reverse transcriptase (RT) assay can then be performed with a DNA primer and a dNTP mixture that are free of capture moiety. With the deactivation of RNase, the resulting cDNA-RNA hybrid can be preserved and captured on a solid phase by binding of the capture moiety to a binding molecule. The amount of incorporated acridinium moieties can be determined upon separation of the hybrid from unreacted acridinium salts.

In another embodiment and as an operational alternative, the DNA primer or RNA template can be immobilized on a solid phase prior to the assay, either by a chemical reaction or through a capture moiety. In this embodiment the solid phase can then be included in the reaction mixture to provide the RNA template or DNA primer, and the reaction proceeds normally. When complete, the solid phase can be removed from the reaction mixture to determine the extent of acridinium ester incorporated into the extending DNA primer.

Triggering of Chemiluminescence

Chemiluminescence of the cDNA copy labeled with multiple acridinium molecules can be triggered in a variety of ways, as is well known in the art. In one embodiment the chemiluminescence is triggered by the addition of two reagents. The first reagent is hydrogen peroxide in dilute acid (e.g., nitric acid), immediately followed by a second reagent containing dilute sodium hydroxide. These reagents oxidize the acridinium ester into an excited state. As the ester returns to ground state, it emits light between 420-430 nm, which is expressed as relative light unites (RLU) and can be detected with a luminometer. In one embodiment the luminometer can automatically inject the trigger solutions and measure light emission. In one embodiment the first trigger solution is hydrogen peroxide in dilute nitric acid, and the second trigger solution is cetyl trimethylammonium chloride in dilute sodium hydroxide.

Each retrovirus is associated with a distinct RT and specific antibodies can be produced against the RT, allowing identification of a specific retrovirus. Thus, the present acridinium-based RT assay is capable of differentiating various retroviruses on the basis of specific binding of RT with antibodies. For example, when an antibody with known RT specificity binds to the RT, cDNA is not produced and no chemiluminescent signal will be detected. Due to the inherent sensitivity and ease of automation for acridinium detection, the present acridinium-based RT assay also permits high throughput screening of anti-retroviral drug and monitoring of anti-retroviral therapy.

The invention will now be described in greater detail with reference to the following non-limiting examples.

### EXAMPLE 1

### Synthesis of Acridinium-Labeled Deoxyuridine Triphosphate

This Example illustrates a method for synthesizing acridinium-labeled deoxyuridine triphosphate. While other dNTPs can also be synthesized using similar techniques, the use of dUTP will result in a more accurate and sensitive assay since it is incorporated into the extending DNA primer by a variety of reverse transcriptases. Additional labeling techniques are found in U.S. Pat. No. 5,185,439 to Arnold et al., which is hereby incorporated by reference in its entirety, including all Tables, Figures, and claims.

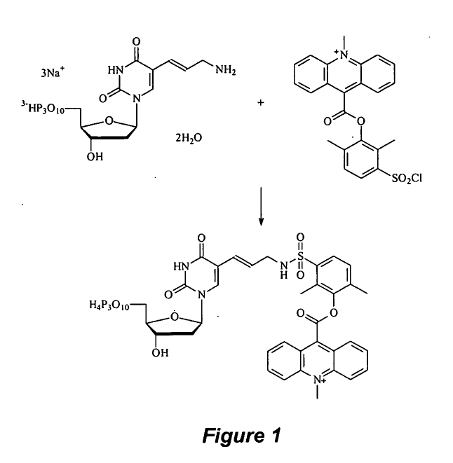

With reference to FIG. 1, lyophilized sodium salt of 5-(3-aminoallyl)-2′-deoxyuridine triphophate dihydrate 1 (AAdUTP, 2.6 mg, 4.16 μmol) is dissolved in 250 μL of 0.5 M sodium bicarbonate/carbonate buffer (pH 9.6) in a vial. Acridinium salt 2 (10.7 mg, 19.2 μmol) is dissolved in 250 μL of anhydrous DMF, and immediately added to the AAdUTP solution with gentle vortex of the AAdUTP solution. The vial is capped and the reaction mixture is allowed to stand overnight at room temperature. The reaction mixture is subject to HPLC purification using the following gradient:
Column: Phenomenex LUNA C18 (2), 5 μm, 4.6×250 mm
Solvent A: 50 mM Triethylamine acetate, pH7.0
Solvent B: Acetonitrile

Wavelengths: 260 nm and 360 nm

| Time (min) | Solvent A (%) | Solvent B (%) |
|---|---|---|
| 0 | 80 | 20 |
| 20 | 60 | 40 |
| 22 | 60 | 40 |
| 23 | 30 | 70 |
| 26 | 30 | 70 |
| 28 | 80 | 20 |
| 32 | 80 | 20 |

Fractions at 15-16 min are collected, combined, and concentrated to dryness in vacuo on a rotary evaporator. The residue is dissolved in water to give a 0.45 mM solution. The purified product is characterized as acridinium-labeled deoxyuridine triphosphate by electrospray ionization mass spectrometry. ESI MS m/z: 950 (M+Na), 927 (M+4H), 847 (M+4H-HPO₃), 829 (M+4H-HPO₃—H₂O), 767 (M+4H-2HPO₃), 749 (M+4H-2HPO₃—H₂O).

### EXAMPLE 2

### Enzyme Degradation and Capillary Electrophoresis Analysis

The integrity of the triphosphate group was assessed by step-wise degradation by alkaline phosphatase, which was monitored by capillary electrophoresis under the following conditions:
Capillary Column: Fused silica, 50 μm×27.0 cm
Buffer: Sodium Borate, pH8.5
Wavelength: 254 nm

Separation program:

| Time | | | Duration |
|---|---|---|---|
| (min) | Function | Value | (min) |
| | Rinse with Buffer | | 2.00 |
| | Pressure Inject | | |
| 0.00 | Separate - Voltage | 15 kV | 7.00 |
| 7.00 | Stop data | | |
| 7.00 | Rinse with 1 M NaOH | | 0.50 |
| 7.50 | Rinse with Water | | 2.00 |
| 9.50 | End | | |

A capillary electrophoregram showed that the peak of the acridinium-labeled deoxyuridine triphosphate at 5.48 min decreased upon addition of alkaline phosphatase with the concurrent formation of the diphosphate and monophosphate species at 5.12 min and 4.30 min, respectively. The triphosphate conjugate eventually disappeared as the degradation proceeded, with the decrease of the peak of the diphosphate intermediate and the concomitant increase of the monophosphate peak. At the end of degradation, the phosphate groups were completely removed, resulting in the final degradation product with a peak at 2.98 min. The triphosphate integrity was thus confirmed by step-wise degradation by alkaline phosphatase and analysis by capillary electrophoresis, ensuring the activity of the acridinium-labeled deoxyuridine triphosphate in reverse transcriptase assay.

### EXAMPLE 3

### Reverse Transcriptase Assay with Recombinant HIV (rHIV) Reverse Transcriptase

A reverse transcriptase assay cocktail was prepared with 50 mM Tris buffer (pH 7.8) that contained 80 mM potassium chloride, 33 mM magnesium chloride, and 11 mM dithiothreitol. The cocktail contained 0.3 μM acridinium-labeled dUTP, 0.3 μM biotin-labeled C11 dUTP, 0.3 μM TTP, and 15 μg/mL poly(rA)-T₁₂₋₁₈ as the template-primer hybrid. rHIV reverse transcriptase (RT) was prepared (as standard and sample) with 50 mM Tris buffer (pH 7.8) that contained 80 mM potassium chloride, 2.5 mM dithiothreitol, 0.75 mM EDTA, and 0.5% Triton X-100. Twenty micro-liters of the assay cocktail was then added to 40 uL of the rHIV standard or sample at 0° C.

The assay mixture thus obtained consisted of the standard or sample rHIV RT, 0.1 μM acridinium-labeled dUTP, 0.1 μM biotin-labeled C11 dUTP, 0.1 μM TTP, 5.0 μg/mL poly(rA)-T₁₂₋₁₈ in 49 mM Tris-HCl as a template, 152 mM potassium chloride, 10 mM magnesium chloride, 0.5 mM EDTA, and 0.3% Triton X-100 at pH 7.8.

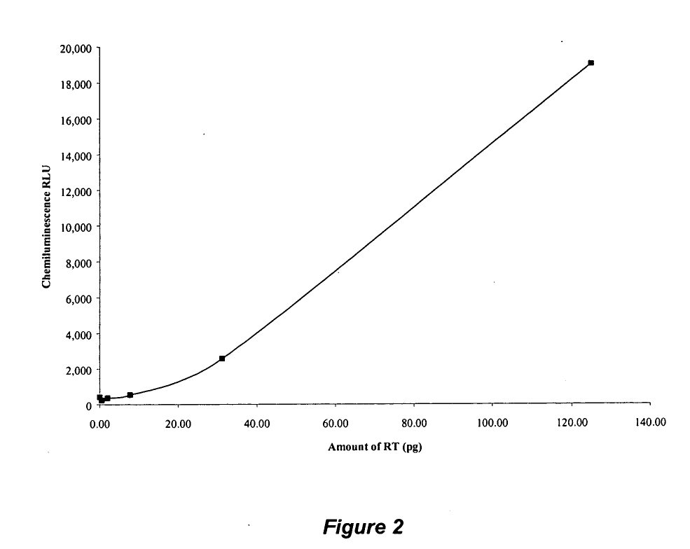

Upon incubation at 37° C. in a sealed reaction vial for appropriate length of time, 5 μL of 0.2 M EDTA was added at 0° C. to stop the assay mixture. The assay mixture was then brought to room temperature, and allowed to stand for 5 min. To 20 μL of magnetic particles (coated with streptavidin) at 2.0 mg/mL in water were added 50 μL of the assay mixture, followed by incubation at room temperature for 5 min. Magnetic separation was applied and the supernatant was removed. The particles were rinsed 3 times with 1 mL of Nichols Advantage® (Quest Diagnostics, Teterboro, N.J.) assay wash concentrate. Deionized water (25 μL) was added to the particles. Relative luminescence unit (RLU) was finally measured for 2 seconds upon triggering the particles with a dilute acid and basic hydrogen peroxide. The data are summarized in following table, and are graphically illustrated in FIG. 2.

| | |
|---|---|
| 0.5 hr | 19 hr |

| STD | RT (pg) | RLU | B/Bo | RLU | B/Bo |
|---|---|---|---|---|---|
| 0 | 0 | 814 | 1.00 | 409 | 1.00 |
| 1 | 0.49 | 833 | 1.02 | 229 | 0.56 |
| 2 | 1.95 | 764 | 0.94 | 354 | 0.87 |
| 3 | 7.81 | 679 | 0.83 | 529 | 1.29 |
| 4 | 31.25 | 2028 | 2.49 | 2566 | 6.27 |
| 5 | 125.00 | 18589 | 22.84 | 18998 | 46.45 |

### EXAMPLE 4

### Synthesis of Biotin-Labeled Nucleotides

Biotin-labeled nucleotides can be synthesized by a variety of methods, one of which is described in this example. In this example two biotin-labeled nucleotide analogs, Bio-4-dUTP and Bio-12-SS-dUPT, are synthesized.

Deoxyuridine 5′-triphosphate is first mercurated at the 5-C and subsequently reacted with allylamine to form 5-(3-amino)allyldeoxyuridine 5′-triphosphate (AA-dUTP). AA-dUTP is purified and reacted with either N-hydroxysuccinimide-activated biotin to form Bio-4-dUTP, or with N-hydroxysuccinimide-activated 2-(biotinamido)ethyl-1,3′-dithiopropionate to form Bio-12-SS-dUTP.

Bio-12-SS-dUTP is a chemically cleavable biotinylated nucleotide analog containing a disulfide bond in the 12-atom linker arm joining biotin to the pyrimidine base. Both biotinylated nucleotide analogs are purified either by ion-exchange chromatography or by ion-pair reverse-phase HPLC. Bio-4-dUTP can be identified by (i) its unique absorbance spectrum, (ii) its co-elution with 3H-Bio-4-dUTP during reverse-phase HPLC, and (iii) its ability to bind to avidin agarose.

As a functional assay for both the synthesis and purification of the biotinylated nucleotide analogs, each nucleotide is incorporated into DNA by nick-translation. The nick-translated DNA is shown to contain biotinylated nucleotides by its ability to bind to biotin-cellulose affinity columns following incubation with soluble avidin. DNA nick-translated in the presence of Bio-12-SS-dUTP is recovered from the biotin-cellulose column following incubation in buffer containing 50 mM dithiothreitol. The susceptibility of the disulfide bond in the linker arm of Bio-12-SS-dUTP to cleavage by dithiothreitol is shown to be unaffected by the presence of avidin bound to the biotin group.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. Modifications therein and other uses will occur to those skilled in the art. These modifications are encompassed within the spirit of the invention and are defined by the scope of the claims.

With reference to the present disclosure, it will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

Other embodiments are set forth within the following claims.

1. A method of detecting the presence of reverse transcriptase in a sample comprising:
contacting the sample with
an RNA template;
a DNA primer;
one or more deoxynucleotide triphosphates labeled with a detectable moiety and, optionally, one or more deoxynucleotide triphosphates not labeled with a detectable moiety;
incubating said reaction mixture under conditions suitable to generate a molecular structure comprising an extended primer comprising said detectable moiety when reverse transcriptase is present in the sample; and
detecting reverse transcriptase in the sample.

2. The method of claim 1 wherein one of said RNA template, DNA primer, deoxynucleotide triphosphate labeled with a detectable moiety, and deoxynucleotide triphosphate not labeled with a detectable moiety, comprises a capture moiety.

3. The method of claim 1 wherein the detecting step comprises detecting the molecular structure.

4. The method of claim 3 wherein the molecular structure comprises the RNA template, the DNA primer, and one or more deoxynucleotide triphosphates labeled with the detectable moiety and, optionally, one or more deoxynucleotide triphosphates not labeled with the detectable moiety.

5. The method of claim 1 wherein the detectable moiety is an acridinium moiety and the detectable signal is the emission of light.

6. The method of claim 1 further comprising separating the molecular structure from the reaction mixture before generating the detectable signal.

7. The method of claim 1 further comprising that unreacted deoxynucleotide triphosphates are removed from the reaction mixture prior to generating the detectable signal.

8. The method of claim 1 wherein the detectable signal is the emission of light and generating the detectable signal comprises the addition of a dilute acid and hydrogen peroxide to the reaction mixture.

9. The method of claim 1 wherein the RNA template comprises homopolymeric and/or heteropolymeric RNA.

10. The method of claim 1 wherein the deoxynucleotide triphosphates comprise dCTP, dGTP, dATP, and dTTP.

11. The method of claim 1 wherein the reaction mixture further comprises one or more divalent metal ions present at a concentration of about 5 mM.

12. The method of claim 11 wherein the divalent metal ions are magnesium and/or manganese present at a concentration of about 5 mM.

13. The method of claim 1 wherein the capture moiety is a hapten.

14. The method of claim 1 wherein the DNA primer comprises a capture moiety.

15. The method of claim 1 wherein the RNA template comprises a capture moiety.

16. The method of claim 1 wherein the detectable moiety is incorporated into the extended primer under conditions suitable to preserve the signal of the detectable moiety.

17. The method of claim 16 wherein the detectable moiety is an acridinium moiety, and the detectable signal is the emission of light.

18. The method of claim 5 wherein the deoxynucleoside triphosphates labeled with the acridinium moiety has the formula:
TP-Sugar-Px-L-Acr
wherein:
TP is a triphosphate group attached to the 5′ position of the sugar;
sugar is a sugar moiety;
Px is a purine, pyrimidine, or 7-deazapurine, wherein Px is attached to the 1′ position of the sugar moiety through the N1 position when Px is a pyrimidine or through the N9 position when Px is a purine or a 7-deazapurine;
L is a linear or branched hydrocarbylene or heterocarbylene linker of at least one carbon atom, wherein L is covalently attached to Acr at one end of L, and at another end to Px through position C5 or C6 of Px when Px is a pyrimidine, or through position C8 of Px when Px is a purine, or through position C7 or C8 of Px when Px is a 7-deazapurine;
Acr is an acridinium moiety; and
the detectable signal is the emission of light.

19. The method of claim 18 wherein the linker, L, is a linear hydrocarbylene or heterocarbylene linker of at least one carbon atom.

20. The method of claim 18 wherein the linker, L, is a linear alkenylene or heteroalkenylene linker containing at least 3 carbon atoms.

21. The method of claim 19 wherein the linker L is —CH₂—CH═CH—CH₂—.

22. The method of claim 19 wherein the linker L is —CH═CH—CH₂—NH—.

23. The method of claim 18 wherein the acridinium moiety is stable under conditions of reverse transcription, and is detectable after incorporation into the extended primer by the emission of light.

24. The method of claim 1 wherein:
the deoxynucleotide triphosphates are labeled with a detectable moiety and with a capture moiety.

25. A molecular structure comprising an RNA template hybridized to an extending DNA primer, which comprises a deoxynucleotide linked to an acridinium moiety.

26. The composition of claim 25 wherein the RNA template further comprises a capture moiety.

27. The composition of claim 25 wherein the DNA molecule further comprises a capture moiety.

28. A kit comprising:
an RNA template;
a DNA primer complementary to a region of the RNA template and of length sufficient to form a stable template-primer hybrid molecule with the RNA template;
a deoxynucleotide triphosphate labeled with a detectable moiety.

29. The kit of claim 28 further comprising buffers for conducting a reverse transcriptase assay.

30. The kit of claim 29 wherein the buffers comprise a divalent metal ion at a concentration of about 5 mM.

31. The kit of claim 28 wherein the detectable moiety is an acridinium moiety.

32. The kit of claim 28 wherein the deoxynucleotide triphosphate further comprises a capture moiety.

33. A method of determining a sub-type of reverse transcriptase present or absent in a sample comprising:
contacting the sample with a binding molecule specific for a sub-type of reverse transcriptase;
contacting the sample with a reaction mixture comprising
an RNA template;
a DNA primer;
a deoxynucleotide triphosphate labeled with a detectable moiety; and
determining whether a molecular structure is generated comprising an extended DNA primer comprising said detectable moiety;
thereby determining whether the sub-type of reverse transcriptase is present or absent in the sample.

34. The method of claim 33 wherein one of said RNA template, DNA primer, and said deoxynucleotide triphosphate labeled with a detectable moiety comprises a capture moiety.

35. The method of claim 33 wherein the binding molecule is immobilized on a surface.

36. The method of claim 33 wherein the detectable moiety is an acridinium moiety.

37. A method of screening for anti-retroviral lead compounds, comprising:
contacting a compound to be tested for anti-retroviral activity with a sample of reverse transcriptase;
contacting the compound and reverse transcriptase with a reaction mixture comprising
an RNA template;
a DNA primer;
a deoxynucleotide triphosphate labeled with a detectable moiety; and
determining whether a molecular structure is generated comprising an extended DNA primer comprising said detectable moiety; and
thereby screening the compound for anti-retroviral activity.

38. The method of claim 37 wherein one of said RNA template, DNA primer, deoxynucleotide triphosphate labeled with a detectable moiety, and deoxynucleotide triphosphate not labeled with a detectable moiety, comprises a capture moiety.

39. A method of detecting the presence of reverse transcriptase in a sample comprising:
contacting the sample with a reaction mixture comprising
an RNA template;
a DNA primer;
one or more deoxynucleotide triphosphates labeled with a detectable moiety;
wherein one of said RNA template or DNA primer is immobilized on a solid phase;
incubating said reaction mixture under conditions suitable to generate a molecular structure comprising an extended primer comprising said detectable moiety when reverse transcriptase is present in said sample; and
detecting reverse transcriptase in said sample.
